(19) United States
(12) Patent Application Publication
(10) Pub. No.: US 2020/0046211 A1
(43) Pub. Date: Feb. 13, 2020

(54) DEVICE FOR DIAGNOSING A TISSUE

(71) Applicant: BIOP - MEDICAL LTD, Ramat Gan (IL)

(72) Inventors: Ilan LANDESMAN, Ramat-Gan (IL); Oz Moshe SEADIA, Bat Yam (IL); Amir GOVRIN, Ramat-Gan (IL)

(73) Assignee: BIOP - MEDICAL LTD, Ramat Gan (IL)

(21) Appl. No.: 16/083,555

(22) PCT Filed: Mar. 9, 2017
(86) PCT No.: PCT/IL2017/050305

§ 371 Date: Sep. 10, 2018

Related U.S. Application Data
(60) Provisional application No. 62/306,122, filed on Mar. 10, 2016.

(51) Int. Cl. A61B 1/06; A61B 1/00; A61B 1/303; A61B 5/00; A61B 1/05

(52) U.S. Cl. CPC A61B 1/0676; A61B 1/00096; A61B 1/0016; A61B 1/0684; A61B 5/0071; A61B 1/05; A61B 1/303

(57) ABSTRACT

A device for diagnosing a tissue is insertable into a patient's cavity. The device includes: (a) a housing; (b) sensors configured for diagnosing tissues within the cavity; (c) light sources having emission spectrum effective for diagnosing the tissue within the cavity; (d) a manipulating element for manipulating the light sources and sensors; (e) at least one display configured for presenting data obtained by the sensors. The manipulating element includes a first member rotatable within the housing around a first axis and a second member rotatable within the first member around a second axis. The second axis is parallel displaced relative to the first axis. The first and second rotatable members are mounted flush with each other and form a front surface, which carries the light sources and sensors facing the tissue to be diagnosed.

### FIELD OF INVENTION

The invention relates to the field of diagnostics of tissue abnormality and in particular to an optical method of tissue disease recognition and a device for implementing the same.

### BACKGROUND OF INVENTION

Cervical cancer is one of the common neoplasms of the female genital tract. Cervical cancer is the second malignancy in women worldwide and is one of the leading causes of women death in the third world. Early diagnosis of abnormal cells in the cervix prevents deterioration into fully cervical cancer and thus reduces morbidity and mortality. The pre-cancerous state is called Squamous Intraepithelial Lesion (SIL), and has two grades: low grade SIL and high grade SIL.

The uterine cervix is very good for screening purposes for several reasons. First, the tumoral changes occur in a specific area, called the transitional zone, around the “external os” (opening of the cervical canal into the vagina). Second, these are slow growing tumors. Third, this area is external in the body and can be easily analyzed by a Gynecologist.

The current screening method, called a Pap smear, has been used for decades. During a Pap smear, a large number of cells, obtained by scraping the cervical epithelium, are smeared onto a slide, or into a liquids tube, which is then fixed and stained for cytologic examination. Unfortunately, the Pap smear is unable to achieve a concurrently high sensitivity and high specificity due to both sampling and analysis errors. Estimations of the sensitivity and specificity of Pap smear screening range from 11-99% and 14-97%, respectively. As used herein, the term sensitivity is defined as the correct classification percentage on pre-cancerous tissue samples, and the term specificity is defined as the correct classification percentage of normal tissue samples. According to the National Cancer Institute (NCI), about 55 million Pap tests are performed each year in the USA. Of these, approximately 3.5 million are abnormal and require medical follow-up. Most of the abnormal tests are in fact falsely indicative of SIL.

Additionally, analyzing Pap smears is extremely labor intensive and requires highly trained professionals. A patient with an abnormal Pap smear indicating the presence of SIL needs to then undergo a diagnostic procedure called colposcopy, which involves colposcopic examination, and if needed biopsy and histology confirmation of the clinical diagnosis. Extensive training is necessary in order for a practitioner to perform colposcopy and its diagnosis accuracy is variable and limited, even in expert hands. Moreover, diagnosis is not immediate.

Thus, it would be desirable to develop a scanning instrument that allows to recognize and to provide mapping of normal and of abnormal tissue areas, which reduces the required skill level of the practitioner interpreting the results and shortens the diagnosis period.

There are several instruments developed last years, which increase sensitivity and a specificity of the diagnostic results. The majority of devices use a combination of different optical effects for the diagnostic. More diagnostic methods allow achieving higher diagnostic accuracy. The number of the methods depend on the specific construction of the apparatus.

In the prior art, there are known local probes used as an addition to colposcope, which are configured for manually screening and cannot provide a map with exact location of suspicious points (see U.S. Pat. No. 8,380,268, U.S. Pat. No. 8,320,650, U.S. Pat. No. 8,005,527, US pre-Grant Publication 20080194969, US pre-Grant Publication 20030013973, PCT Publication WO 2014007759 and J. A. Tidy et al, Accuracy of detection of high-grade cervical intraepithelial neoplasia using electrical impedance spectroscopy with colposcopy. BJOG: An International Journal of Obstetrics & Gynaecology, 120, No. 4, pp. 400-411, March 2013),

For example, U.S. Pat. No. 8,005,527 discloses a system and method for the in situ discrimination of healthy and diseased tissue. A fiberoptic based probe is employed to direct ultraviolet illumination onto a tissue specimen and to collect the fluorescent response radiation. The response radiation is observed at three selected wavelengths, one of which corresponds to an isosbestic point. In one example, the isosbestic point occurs at about 431 nm. The intensities of the observed signals are normalized using the 431 nm intensity. A score is determined using the ratios in a discriminant analysis. The tissue under examination is resected or not, based on the diagnosis of disease or health, according to the outcome of the discriminant analysis.

U.S. Pat. No. 6,590,651 discloses an apparatus and method embodying the invention include utilizing a device with a limited number of interrogation devices to accomplish a large number of measurements on a target tissue. An instrument embodying the invention includes a plurality of detection devices that are arranged in a predetermined pattern on a tissue contacting face of the instrument. The face of the instrument is located adjacent the target tissue, and a plurality of tissue characteristic measurement are simultaneously conducted. The detection devices are moved to a new position, preferably without moving the tissue contacting face, and a second plurality of tissue characteristic measurements are simultaneously conducted. By conducting a series of measurements cycles in this manner, the ultimate resolution of the device is increased, while still obtaining a given resolution, which reduces potential cross-talk errors. Further, a plurality of tissue characteristics are simultaneously obtained from locations spaced across the target tissue during each measurement cycle.

US pre-Grant publication 2012232404 discloses a method and apparatus that interrogate, receive, and analyze full emission spectra for at least one fluorescence excitation wavelength and for at least one reflectance measurement to determine tissue characteristics and correlate same to photographic images. Further, the system and method accomplish this measurement rapidly by increasing the light throughput by integrating optics into a hand held unit and avoiding the need for a coherent fiber optic bundle being used. The method includes illuminating a first portion of a target tissue with optical energy, forming a first image of the target tissue, illuminating a second portion of the target tissue with optical energy, performing spectroscopic measurements on optical energy reflected and/or emitted by the target tissue upon illumination of the second portion of the target tissue with optical energy, and determining tissue characteristics of the target tissue based on the results of the spectroscopic measurements.

U.S. Pat. No. 7,127,282 discloses a method and a system provided for discriminating between healthy cervical tissue and pathologic cervical tissue based on the fluorescence response of the tissue to laser excitation (LIF) and the backscatter response to illumination by white light (in the spectral range of 360 to 750 nm). Combining LIF and white light responses, as well as evaluating a spatial correlation between proximate cervical tissue sites in conjunction with a statistically significant “distance” algorithm, such as the Mahalanobis distance between data sets, can improve the discrimination between normal and abnormal tissue. The results may be displayed in the form of a map of the cervix representing the suspected pathology.

All abovementioned prior art documents do not teach any coloposcope. U.S. Pat. No. 5,623,932 discloses an apparatus and in vivo methods to distinguish normal and abnormal cervical tissue and to detect cervical intraepithelial neoplasia (CIN) in a diagnostic cervical tissue sample. Induced fluorescence intensity spectra from known normal cervical tissue and a diagnostic tissue sample are obtained from the same patient. Peak fluorescence intensity values for normal tissue samples are averaged, as are slope measurements from predetermined portions of spectra induced in both known normal cervical tissue and the diagnostic tissue sample. Peak fluorescence intensities of diagnostic tissue spectra are divided by average peak fluorescence intensity values for normal tissue in the same patient to yield relative peak fluorescence intensity values. Normal and abnormal cervical tissues are distinguished using a predetermined empirical discriminant function of slope measurements derived from normal tissue spectra and relative peak fluorescence intensity measurements in the same patient. CIN is distinguished from tissue with human papilloma virus infection or inflammation using a predetermined empirical discriminant function of average slope measurements on spectra from known normal tissue and slope measurements on a diagnostic tissue spectrum It is known in the art that, during a testing procedure, a patient cannot to be absolutely immobilized and moves relative to the probe. To hold the obtained data down, the patient's displacement should be measured and taken into consideration. Thus, there is a long-felt and unmet need to provide a device for colposcopy which enables measuring displacement of a tissue to be diagnosed and reconsidering the obtained data in this context.

Another long-felt and unmet need to provide a device for colposcopy which enables mapping the cervix in a multi-instrumental manner in order to decrease a chance of decease recognition.

### SUMMARY OF THE INVENTION

It is hence one object of the invention to disclose a device for diagnosing a tissue. The aforesaid device is insertable into a patient's cavity. The device comprises: (a) a housing; (b) at least one sensor configured for diagnosing said tissue within the cavity; (c) at least one light source having emission spectrum effective for diagnosing the tissue within the cavity; (d) means for manipulating the at least one light source and at least one sensor; (e) displaying means configured for presenting data obtained by the at least one sensor.

It is a core purpose of the invention to provide the manipulating means further comprising a first member rotatable within the housing around a first axis and a second member rotatable within the first member around a second axis. The second axis is parallel displaced from the first axis. The first and second rotatable members are mounted flush with each other and form a front surface, which carries the at least one light source and the at least one sensor facing the tissue to be diagnosed.

Another object of the invention is to disclose the housing which is of a tubular shape. The housing has a longitudinal axis.

A further object of the invention is to disclose the first rotatable member mounted concentrically with the housing axis.

A further object of the invention is to disclose at least one sensor disposed on the front surface of the second rotatable member at a distance r from the second axis; the second axis is parallel dislodged from the first axis by distance r.

A further object of the invention is to disclose at least one of the first and second rotatable members comprising a cogwheel circumferentially embracing the rotatable member; the cogwheel is coupled with a driving gear mechanically connected to a drive.

A further object of the invention is to disclose the drive which is an electric motor.

A further object of the invention is to disclose at least one light source selected from the group consisting of a white light emitting diode, a coherent laser light source in visual or near infrared ranges, a UV light source effective for auto-fluorescence excitation and any combination thereof.

A further object of the invention is to disclose at least one sensor selected from the group consisting of a panoramic camera, a camera for capturing scattering patterns, a close-up camera, an optical fiber connected to a spectrometer and any combination thereof.

A further object of the invention is to disclose the device comprising a multifunctional passage for sampling the tissue at the suspicious locations or administering medicines or other materials into the cavity.

A further object of the invention is to disclose the device comprising a sensor of mutual displacement of said tissue area to be diagnosed and the device.

A further object of the invention is to disclose a method of diagnosing a tissue in a patient's cavity. The aforesaid method comprises the steps of: (a) providing a device comprising: (i) a housing; (ii) at least one sensor configured for diagnosing the tissue within the cavity; the sensor is selected from the group consisting of a panoramic camera, a camera for capturing scattering patterns, a close-up camera, an optical fiber connected to a spectrometer and any combination thereof; (iii) at least one light source having emission spectrum effective for diagnosing the tissue within the cavity; the light source selected from the group consisting of a white light laser emitting diode, a coherent laser light source, a UV light source effective for auto-fluorescence excitation and any combination thereof; (iv) means for manipulating the at least one light source and at least one sensor; (v) displaying means configured for presenting data obtained by the at least one sensor; the manipulating means further comprises a first member rotatable within the housing around a first axis and a second member rotatable within the first member around a second axis; the second axis is parallel displaced from the first axis; the first and second rotatable members are mounted flush with each other and form a front surface, which carries the at least one light source and at least one sensor facing the tissue to be diagnosed; (b) inserting the device into the patient's cavity; (c) capturing a panoramic image of a tissue area to be diagnosed; (d) detecting a target area which is suspicious for malignancy; (e) marking the target area in images presented by the displaying means; (f) navigating the device to the target area; (g) interrogating tissue data by means of the at least one sensor.

It is another core purpose of the invention to provide the step of interrogating tissue data is performed by angular displacement of the first and second member relative to the housing and to each other in a successive manner

A further object of the invention is to disclose the step of inserting the device into the patient's cavity comprising inserting the housing of a tubular shape along longitudinal axis thereof.

A further object of the invention is to disclose the step of interrogating tissue data comprising rotating the first member mounted concentrically with the housing axis.

A further object of the invention is to disclose the step of interrogating tissue data performed by the at least one sensor disposed on the front surface of the second rotatable member at a distance r from the second axis; the second axis is parallel dislodged from the first axis by distance r.

A further object of the invention is to disclose the step of interrogating tissue data comprising a sub-step of rotating at least one of the first and second rotatable members by a cogwheel circumferentially embracing the at least one of the first and second rotatable member; the cogwheel is coupled with a driving gear mechanically connected to a drive.

A further object of the invention is to disclose the sub-step of rotating at least one of the first and second rotatable members performed by an electric motor.

A further object of the invention is to disclose the method comprising a step of sampling the tissue at the suspicious locations or administering medicines or other materials into the cavity via a multifunctional passage.

A further object of the invention is to disclose the method comprising a step of measuring of mutual displacement of the tissue area to be diagnosed and the device.

A further object of the invention is to disclose the step of detecting a marked target area comprising speeded up robust features procedure.

A further object of the invention is to disclose the step of tracking and marking the target area comprising Kanade-Lucas-Tomasi tracker procedure.

### BRIEF DESCRIPTION OF THE DRAWINGS

In order to understand the invention and to see how it may be implemented in practice, a plurality of embodiments is adapted to now be described, by way of non-limiting example only, with reference to the accompanying drawings, in which

FIGS. 1a and 1b are schematic diagrams illustrating angular displacement of first and second rotatable members;

FIG. 2 is an exploded isometric view of a gear arrangement of a device for diagnosing a tissue;

FIG. 3 is a conceptual schematic front view of a device for diagnosing a tissue; and

FIG. 4 is a front view of an exemplar embodiment of a device for diagnosing a tissue.

### DETAILED DESCRIPTION OF THE INVENTION

The following description is provided, so as to enable any person skilled in the art to make use of said invention and sets forth the best modes contemplated by the inventor of carrying out this invention. Various modifications, however, are adapted to remain apparent to those skilled in the art, since the generic principles of the present invention have been defined specifically to provide a device for diagnosing a tissue and a method of using the same.

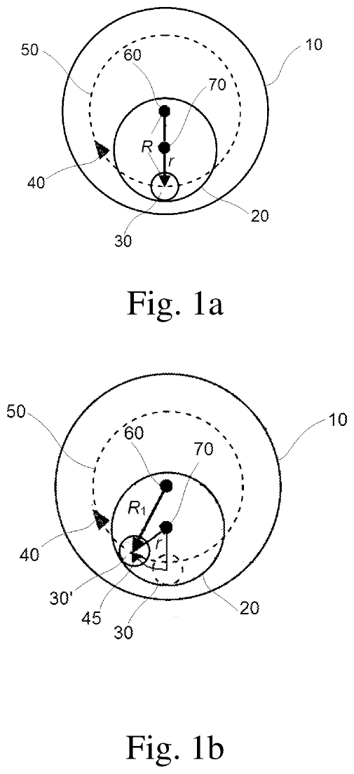

Reference is now to FIGS. 1a and 1b presenting two front views of the diagnosing device which show two exemplar positions rotatable members 10 and 20. First member 10 is rotatable around axis 60 within a housing (not shown) while second member 20 is rotatable around axis 70. Rotatable members 10 and 20 are mounted flush with each other and form a front surface. Numeral 30 refers to a sensor carried by second rotatable member 20. Sensor 30 is at distance r from axis 70. If axes 60 and 70 are at the same distance r, mutual rotation of first and second rotatable members 10 and 20 provide positioning sensor 30 within circle 50 of radius R. Thus if R=2r, sensor 30 can be positioned in any point within circle 50. FIG. 1a demonstrates an exemplar mutual position of first and second rotatable members 10 and 20. Sensor 30 is at distance R₁ from axis 60.

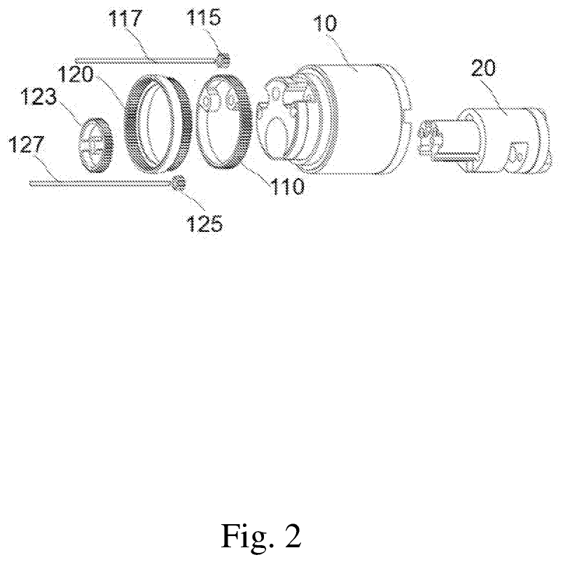

Reference is now made to FIG. 2, presenting a gear arrangement of the device for diagnosing a tissue. Specifically, shafts 117 and 127 provided with gears 115 and 125, respectively, are rotatable by a common drive or two separate drives (for example, electric step motors). Gear 115 is coupled with cogwheel 110 circumferentially embracing first rotatable member 10. Thus, rotation from shaft 117 is transferred to first rotatable member 10. Concerning second rotatable member 20, gear 125 is coupled with an external side of two-side cogwheel 120. Cogwheel 123 circumferentially embracing second rotatable member 20 is coupled with an internal side of cogwheel 120. Therefore, rotation from shaft 127 is transferred to second rotatable member 20 via the following gear assembly: elements 125-120-123.

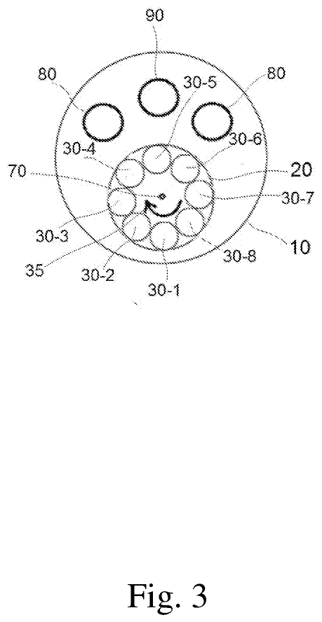

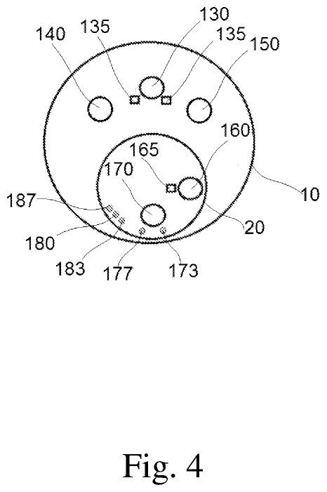

Reference is now made to FIG. 3 showing a conceptual schematic front view of a device for diagnosing a tissue. In FIG. 3, second member is rotatable around axis 70 in an exemplar direction 35 such that any of sensors 30-1 to 30-8 can be positioned in a location of interest. Numeral 80 refers to white-light LEDs illuminating a tissue to be examined (not shown) such that a tissue image can be captured by panoramic camera 90. Reference is now made to FIG. 4 showing an exemplar embodiment of a device for diagnosing a tissue. Specifically, when the device of the present invention faces the cervix, its panoramic image is captured by camera 130 under illumination provided by white-light sources 135. Suspicious locations are marked within the panoramic image. The device is inserted till full contact with the cervix and the suspicious locations are examined by a plurality of sensors which are described below. The device is provided with a sensor of mutual displacement of the cervix and the device of the present invention. As mentioned above, the patient cannot to be absolutely immobilized and moves relative to the probe. Sensor 140 is designed for measuring displacement of a tissue to be diagnosed and remark the suspicious locations at the cervix. Numeral 150 refers to a multifunctional passage used for tissue sampling at the suspicious locations of the cervix or administering medicines or other materials into the cavity (not shown). Second rotatable member 20 is provided with microscopic camera 160 and white-light source 165 for capturing a more detailed microscopic image. Sensor 170 is configured for capturing scattering pattern obtained under illumination by laser sources 173 and 177 emitting in near infrared and visible spectral ranges, respectively. Near infrared radiation has deeper penetration depth and affected by stroma of the cervix. The short wavelength penetrates for a smaller depth and mostly affected by epithelial layer of the tissue. The ratio of scattered light intensity distributions can be one of indicators of tissue abnormal conditions.

Bore 180 accommodates an optical fiber connected to a spectrometer (not shown) for spectral analysis. Light from white-light source 183 reflected by the cervix tissue and auto-fluorescence excited by light source 187 are conducted to the spectrometer. The workflow of the tracking algorithm includes the following four steps:
1. Capturing a panoramic image by panoramic camera 130 of the cervix.
2. Marking a target point for examination.
3. Assisting navigation of the device to the target point with dynamic marking of the target point in video flow captured by the panoramic camera;
4. Determining live scan coordinates according to obtained data of mutual displacement of the device after full contact.

The workflow of processing is the following:
1. Defining a region of interest (ROI) by detecting Speeded Up Robust Features (SURF) within it. When correspondence between SURF feature and obtained live video flow is within predetermined tolerance ROI is marked. Other feature extraction algorithms are also in the scope of the present invention.
2. The marked ROI is tracked in live video flow by the Kanade-Lucas-Tomasi (KLT) procedure (see C. Tomasi et al, Detection and Tracking of Point Features, Carnegie Mellon University Technical Report CMU-CS-91-132, April 1991). The algorithm tracks corner points (J. Shi et al, Good Features to Track, Proceedings of the IEEE Conference on Computer Vision and Pattern Recognition. June 1994, pp. 593-600) around the selected target. In order to handle larger displacements, a pyramid representation of the two frames is used. The tracking algorithm provides geometric transformation from frame to frame by computing a new location of the target point. and displaying this new location on screen for the user. If the target point is lost due to large movements of the user, the algorithm goes back to the target detection stage. When target is redetected we move back to the tracking stage. This process is repeated till full contact with the cervix wall is reached. After full contact, measurement of lateral displacement of tissue to be examined relative to the device is performed. The obtained mutual displacement data are used for updating the position of the scanning coordinates.

According to the present invention, a device for diagnosing a tissue is disclosed. The aforesaid device is insertable into a patient's cavity. The device comprises: (a) a housing; (b) at least one sensor configured for diagnosing said tissue within the cavity; (c) at least one light source having emission spectrum effective for diagnosing the tissue within the cavity; (d) means for manipulating the at least one light source and at least one sensor; (e) displaying means configured for presenting data obtained by the at least one sensor.

It is a feature purpose of the invention to provide the manipulating means further comprising a first member rotatable within the housing around a first axis and a second member rotatable within the first member around a second axis. The second axis is parallel displaced from the first axis. The first and second rotatable members are mounted flush with each other and form a front surface, which carries the at least one light source and the at least one sensor facing the tissue to be diagnosed.

According to one embodiment of the present invention, the housing is of a tubular shape. The housing has a longitudinal axis.

According to another embodiment of the present invention, the first rotatable member is mounted concentrically with the housing axis.

According to a further embodiment of the present invention, at least one sensor is disposed on the front surface of the second rotatable member at a distance r from the second axis; the second axis is parallel dislodged from the first axis by distance r.

According to a further embodiment of the present invention, at least one of the first and second rotatable members comprises a cogwheel circumferentially embracing the rotatable member; the cogwheel is coupled with a driving gear mechanically connected to a drive.

According to a further embodiment of the present invention, the drive is an electric motor.

According to a further embodiment of the present invention, at least one light source is selected from the group consisting of a white light emitting diode, a coherent laser light source in visual or near infrared ranges, a UV light source effective for auto-fluorescence excitation and any combination thereof.

According to a further embodiment of the present invention, at least one sensor is selected from the group consisting of a panoramic camera, a camera for capturing scattering patterns, a close-up camera, an optical fiber connected to a spectrometer and any combination thereof.

According to a further embodiment of the present invention, the device comprises a multifunctional passage for sampling the tissue at the suspicious locations or administering medicines or other materials into the cavity.

According to a further embodiment of the present invention, the device comprises a sensor of mutual displacement of said tissue area to be diagnosed and the device.

According to a further embodiment of the present invention, a method of diagnosing a tissue in a patient's cavity is disclosed. The aforesaid method comprises the steps of: (a) providing a device comprising: (i) a housing; (ii) at least one sensor configured for diagnosing the tissue within the cavity; the sensor is selected from the group consisting of a panoramic camera, a camera for capturing scattering patterns, a close-up camera, an optical fiber connected to a spectrometer and any combination thereof; (iii) at least one light source having emission spectrum effective for diagnosing the tissue within the cavity; the light source selected from the group consisting of a white light laser emitting diode, a coherent laser light source, a UV light source effective for auto-fluorescence excitation and any combination thereof; (iv) means for manipulating the at least one light source and at least one sensor; (v) displaying means configured for presenting data obtained by the at least one sensor; the manipulating means further comprises a first member rotatable within the housing around a first axis and a second member rotatable within the first member around a second axis; the second axis is parallel displaced from the first axis; the first and second rotatable members are mounted flush with each other and form a front surface, which carries the at least one light source and at least one sensor facing the tissue to be diagnosed; (b) inserting the device into the patient's cavity; (c) capturing a panoramic image of a tissue area to be diagnosed; (d) detecting a target area which is suspicious for malignancy; (e) marking the target area in images presented by the displaying means; (f) navigating the device to the target area; (g) interrogating tissue data by means of the at least one sensor.

It is another core feature of the invention to provide the step of interrogating tissue data is performed by angular displacement of the first and second member relative to the housing and to each other in a successive manner

According to a further embodiment of the present invention, the step of inserting the device into the patient's cavity comprises inserting the housing of a tubular shape along longitudinal axis thereof.

According to a further embodiment of the present invention, the step of interrogating tissue data comprises rotating the first member mounted concentrically with the housing axis.

According to a further embodiment of the present invention, the step of interrogating tissue data is performed by the at least one sensor disposed on the front surface of the second rotatable member at a distance r from the second axis; the second axis is parallel dislodged from the first axis by distance r.

According to a further embodiment of the present invention, the step of interrogating tissue data comprises a sub-step of rotating at least one of the first and second rotatable members by a cogwheel circumferentially embracing the at least one of the first and second rotatable member; the cogwheel is coupled with a driving gear mechanically connected to a drive.

According to a further embodiment of the present invention, the sub-step of rotating at least one of the first and second rotatable members is performed by an electric motor.

According to a further embodiment of the present invention, the method comprises a step of sampling the tissue at the suspicious locations or administering medicines or other materials into the cavity via a multifunctional passage.

According to a further embodiment of the present invention, the method comprises a step of measuring of mutual displacement of the tissue area to be diagnosed and the device.

According to a further embodiment of the present invention, the step of detecting a marked target area comprises speeded up robust features procedure.

According to a further embodiment of the present invention, the step of tracking and marking the target area comprises Kanade-Lucas-Tomasi tracker procedure.

1. A device for diagnosing a tissue; said device insertable into a patient's cavity; said device comprising:
a. a housing;
b. at least one sensor configured for diagnosing said tissue within said cavity;
c. at least one light source having emission spectrum effective for diagnosing said tissue within said cavity;
d. means for manipulating said at least one light source and at least one sensor;
e. displaying means configured for presenting data obtained by said at least one sensor;
wherein said manipulating means further comprises a first member rotatable within said housing around a first axis and a second member rotatable within said first member around a second axis; said second axis is parallel displaced from said first axis; said first and second rotatable members are mounted flush with each other and form a front surface, which carries said at least one light source and said at least one sensor facing said tissue to be diagnosed.

2. The device according to claim 1, wherein said housing is of a tubular shape; said housing has a longitudinal axis.

3. The device according to claim 2, wherein said first rotatable member is mounted concentrically with said housing axis.

4. The device according to claim 1, wherein said at least one sensor is disposed on said front surface of said second rotatable member at a distance r from said second axis; said second axis is parallel dislodged from said first axis by distance r.

5. The device according to claim 1, wherein at least one of said first and second rotatable members comprises a cogwheel circumferentially embracing said rotatable member; said cogwheel is coupled with a driving gear mechanically connected to a drive.

6. The device according to claim 5, wherein said drive is an electric motor.

7. The device according to claim 1, wherein said at least one light source is selected from the group consisting of a white light emitting diode, a coherent laser light source in visual or near infrared ranges, a UV light source effective for auto-fluorescence excitation and any combination thereof.

8. The device according to claim 1, wherein said at least one sensor is selected from the group consisting of a panoramic camera, a camera for capturing scattering patterns, a close-up camera, an optical fiber connected to a spectrometer and any combination thereof.

9. The device according to claim 1 comprising a multifunctional passage for sampling said tissue at the suspicious locations or administering medicines or other materials into the cavity.

10. The device according to claim 1 comprising a sensor of mutual displacement of said tissue area to be diagnosed and the device.

11. A method of diagnosing a tissue in a patient's cavity; said method comprising the steps of:
a. providing a device comprising:
i. a housing;
ii. at least one sensor configured for diagnosing said tissue within said cavity; said sensor is selected from the group consisting of a panoramic camera, a camera for capturing scattering patterns, a close-up camera, an optical fiber connected to a spectrometer and any combination thereof;
iii. at least one light source having emission spectrum effective for diagnosing said tissue within said cavity; said light source selected from the group consisting of a white light laser emitting diode, a coherent laser light source, a UV light source effective for auto-fluorescence excitation and any combination thereof;
iv. means for manipulating said at least one light source and at least one sensor;
v. displaying means configured for presenting data obtained by said at least one sensor;
said manipulating means further comprises a first member rotatable within said housing around a first axis and a second member rotatable within said first member around a second axis;
said second axis is parallel displaced from said first axis; said first and second rotatable members are mounted flush with each other and form a front surface, which carries said at least one light source and at least one sensor facing said tissue to be diagnosed;
b. inserting said device into said patient's cavity;
c. capturing a panoramic image of a tissue area to be diagnosed;
d. detecting a target area which is suspicious for malignancy;
e. marking said target area in images presented by said displaying means;
f. navigating said device to said target area;
g. interrogating tissue data by means of said at least one sensor;
wherein said step of interrogating tissue data is performed by angular displacement of said first and second member relative to said housing and to each other in a successive manner

12. The method according to claim 11, wherein said step of inserting said device into said patient's cavity comprises inserting said housing of a tubular shape along longitudinal axis thereof.

13. The method according to claim 12, wherein said step of interrogating tissue data comprises rotating said first member mounted concentrically with said housing axis.

14. The method according to claim 11, wherein said step of interrogating tissue data is performed by said at least one sensor disposed on said front surface of said second rotatable member at a distance r from said second axis; said second axis is parallel dislodged from said first axis by distance r.

15. The method according to claim 11, wherein said step of interrogating tissue data comprises a sub-step of rotating at least one of said first and second rotatable members by a cogwheel circumferentially embracing said at least one of said first and second rotatable member; said cogwheel is coupled with a driving gear mechanically connected to a drive.

16. The method according to claim 15, wherein said sub-step of rotating at least one of said first and second rotatable members is performed by an electric motor.

17. The method according to claim 11 comprising a step of sampling said tissue at the suspicious locations or administering medicines or other materials into the cavity via a multifunctional passage.

18. The method according to claim 11 comprising a step of measuring of mutual displacement of said tissue area to be diagnosed and the device.

19. The method according to claim 11, wherein said step of detecting a marked target area comprises speeded up robust features procedure.

20. The method according to claim 11, wherein said step of tracking and marking said target area comprises Kanade-Lucas-Tomasi tracker procedure.
